(19) United States
(12) Patent Application Publication
(10) Pub. No.: US 2023/0172659 A1
(43) Pub. Date: Jun. 8, 2023

(54) SYSTEMS AND METHODS FOR ENERGIZING ELECTROPORATION CATHETERS

(71) Applicant: St. Jude Medical, Cardiology Division, Inc., St. Paul, MN (US)

(72) Inventors: Eric Olson, Maplewood, MN (US); Greg Olson, Elk River, MN (US)

(21) Appl. No.: 17/984,409

(22) Filed: Nov. 10, 2022

Related U.S. Application Data
(60) Provisional application No. 63/278,603, filed on Nov. 12, 2021.

(51) Int. Cl. A61B 18/14; A61B 18/12; A61B 5/06; G16H 20/70

(52) U.S. Cl. CPC A61B 18/1492; A61B 5/065; A61B 18/1206; G16H 20/70; A61B 2018/00577

(57) ABSTRACT

An apparatus for controlling an electroporation catheter is provided. The electroporation catheter includes a distal end, a proximal end, at least one spline extending from the distal end to the proximal end, and a plurality of electrodes arranged on the at least one spline. The apparatus includes a pulse generator coupled to the electroporation catheter, and a computing device coupled to the pulse generator, the computing device operable to control the pulse generator to selectively energize the plurality of electrodes on the electroporation catheter to form an energization pattern.

### CROSS-REFERENCE TO RELATED APPLICATIONS

This application claims priority to provisional application serial No. 63/278,603, filed Nov. 12, 2021, which is incorporated herein by reference in its entirety.

### BACKGROUND

It is generally known that ablation therapy may be used to treat various conditions afflicting the human anatomy. For example, ablation therapy may be used in the treatment of atrial arrhythmias. When tissue is ablated, or at least subjected to ablative energy generated by an ablation generator and delivered by an ablation catheter, lesions form in the tissue. Electrodes mounted on or in ablation catheters are used to create tissue necrosis in cardiac tissue to correct conditions such as atrial arrhythmia (including, but not limited to, ectopic atrial tachycardia, atrial fibrillation, and atrial flutter).

Arrhythmia (i.e., irregular heart rhythm) can create a variety of dangerous conditions including loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a non-thermal ablation technique that involves applying strong electric-fields that induce pore formation in the cellular membrane. The electric field may be induced by applying a relatively short duration pulse which may last, for instance, from a nanosecond to several milliseconds. Such a pulse may be repeated to form a pulse train. When such an electric field is applied to tissue in an in vivo setting, the cells in the tissue are subjected to trans-membrane potential, which opens the pores on the cell wall. Electroporation may be reversible (i.e., the temporally-opened pores will reseal) or irreversible (i.e., the pores will remain open). For example, in the field of gene therapy, reversible electroporation (i.e., temporarily open pores) is used to transfect high molecular weight therapeutic vectors into the cells. In other therapeutic applications, a suitably configured pulse train alone may be used to cause cell destruction, for instance by causing irreversible electroporation.

For example, pulsed field ablation (PFA) may be used to perform instantaneous pulmonary vein isolation (PVI). PFA generally involves delivering high voltage pulses from electrodes disposed on a catheter. For example, voltage pulses may range from less than about 500 volts to about 2400 volts or higher. These fields may be applied between pairs of electrodes (bipolar therapy) or between one or more electrodes and a return patch (monopolar therapy).

### BRIEF SUMMARY OF THE DISCLOSURE

In one aspect an apparatus for controlling an electroporation catheter is provided. The electroporation catheter includes a distal end, a proximal end, at least one spline extending from the distal end to the proximal end, and a plurality of electrodes arranged on the at least one spline. The apparatus includes a pulse generator coupled to the electroporation catheter, and a computing device coupled to the pulse generator, the computing device operable to control the pulse generator to selectively energize the plurality of electrodes on the electroporation catheter to form an energization pattern.

In another aspect, a method for controlling an ablation system including an ablation catheter, a pulse generator coupled to the ablation catheter, and a computing device coupled to the pulse generator is provided. The method includes identifying, using the computing device, at least one target ablation location on an anatomy of a patient, tracking, using the computing device, movement of the ablation catheter through the patient, the ablation catheter including a plurality of electrodes, determining, based on the tracking, using the computing device, that at least one electrode of the plurality of electrodes is proximate the at least one target ablation location, and selectively energizing, using the pulse generator, the at least one electrode to ablate the at least one target ablation location.

In yet another aspect an apparatus for controlling an electroporation catheter is provided. The electroporation catheter includes a distal end, a proximal end, a plurality of splines extending from the distal end to the proximal end, and a plurality of electrodes arranged on the plurality of splines, the plurality of splines and the plurality of electrodes forming a grid assembly. The apparatus includes a pulse generator coupled to the electroporation catheter, and a computing device coupled to the pulse generator, the computing device operable to control the pulse generator to selectively energize the plurality of electrodes on the grid assembly to form an energization pattern.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic and block diagram view of a system for electroporation therapy.

FIG. 2 is a side view of one embodiment of a grid assembly that may be used with the catheter shown in FIG. 1.

FIG. 3 is an image showing the grid assembly of FIG. 2 positioned within a patient’s heart.

FIGS. 4A-4C illustrate a plurality of example energization patterns using the grid assembly shown in FIG. 2.

FIGS. 5A and 5B illustrate a plurality of example energization patterns using the grid assembly shown in FIG. 2.

FIGS. 6A and 6B are perspective views of one embodiment of a basket assembly that may be used with the catheter shown in FIG. 1.

FIGS. 7A-7C are views of another embodiment of a basket assembly that may be used with the catheter shown in FIG. 1.

FIG. 8 is a flowchart of an example method for generating a lesion set that may be used with the system shown in FIG. 1.

FIG. 9 is a schematic diagram of one embodiment of a switching architecture that may be used with the system shown in FIG. 1.

### DETAILED DESCRIPTION OF THE DISCLOSURE

The systems and methods described herein are directed to an apparatus for controlling an electroporation catheter is provided. The electroporation catheter includes a distal end, a proximal end, at least one spline extending from the distal end to the proximal end, and a plurality of electrodes arranged on the at least one spline. The apparatus includes a pulse generator coupled to the electroporation catheter, and a computing device coupled to the pulse generator, the computing device operable to control the pulse generator to selectively energize the plurality of electrodes on the electroporation catheter to form an energization pattern.

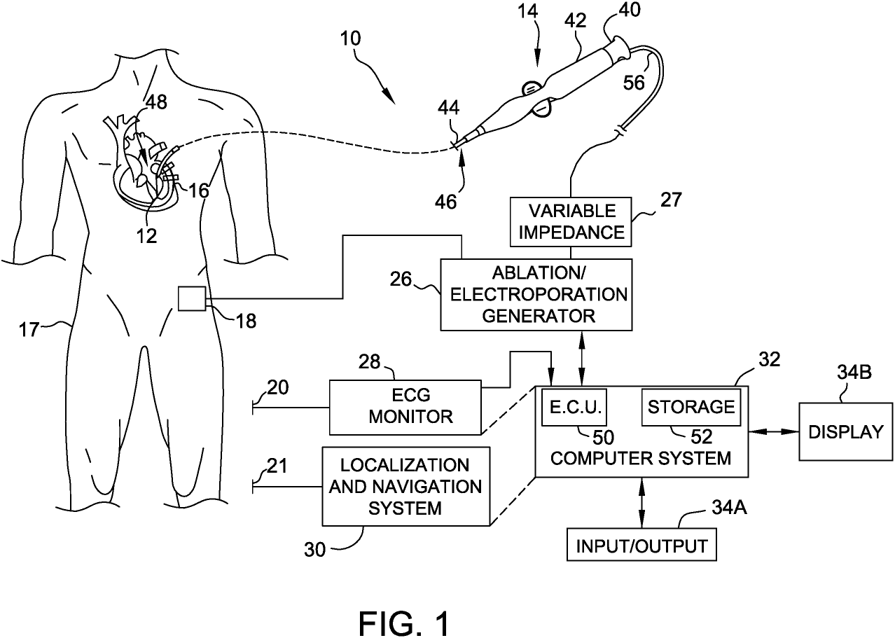

FIG. 1 is a schematic and block diagram view of a system 10 for electroporation therapy. In general, system 10 includes a catheter electrode assembly 12 disposed at a distal end 48 of a catheter 14. As used herein, “proximal” refers to a direction toward the end of the catheter near the clinician and “distal” refers to a direction away from the clinician and (generally) inside the body of a patient. The electrode assembly includes one or more individual, electrically-isolated electrode elements. Each electrode element, also referred to herein as a catheter electrode, is individually wired such that it can be selectively paired or combined with any other electrode element to act as a bipolar or a multi-polar electrode.

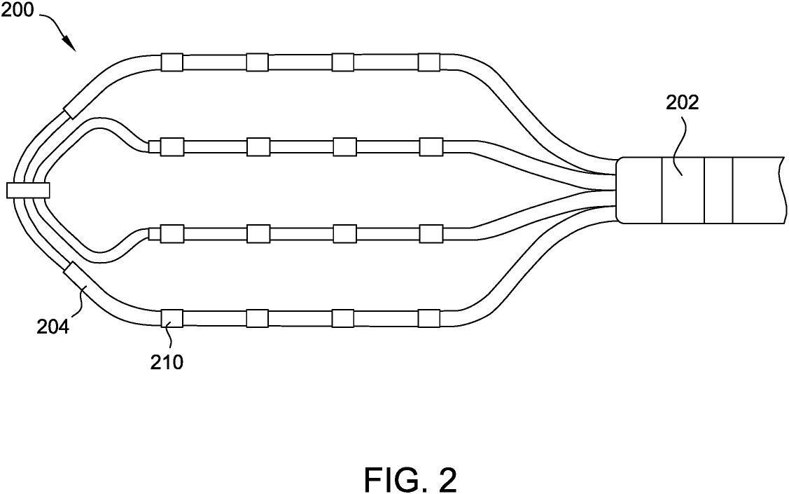

System 10 may be used for irreversible electroporation (IRE) to destroy tissue. In particular, system 10 may be used for electroporation-induced therapy that includes delivering electrical current in such a manner as to directly cause an irreversible loss of plasma membrane (cell wall) integrity leading to its breakdown and cell necrosis. This mechanism of cell death may be viewed as an “outside-in” process, meaning that the disruption of the outside wall of the cell causes detrimental effects to the inside of the cell. Typically, for classical plasma membrane electroporation, electric current is delivered as a pulsed electric field in the form of short-duration pulses (e.g., having a 100 nanosecond (ns) to 100 microsecond (µs) duration) between closely spaced electrodes capable of delivering an electric field strength of about 0.1 to 10.0 kilovolts/centimeter (kV/cm). System 10 may be used with a grid catheter such as that depicted in FIG. 2, for example, for high output (e.g., high voltage and/or high current) electroporation procedures. Alternatively, system 10 may be used with any suitable catheter configuration.

In one embodiment, all electrodes of the catheter deliver an electric current simultaneously. Alternatively, in other embodiments, stimulation is delivered selectively (e.g., between pairs of electrodes) on the catheter. For example, in some embodiments, the catheter includes a plurality of splines, each spline including a plurality of electrodes. In such embodiments, electrodes on one spline may be selectively activated, and electrodes on an adjacent (or other) spline function as an energy return (or sink). Further, in the embodiments described herein, the electrodes may be switchable between being connected to a 3D mapping system and being connected to an electroporation generator.

Irreversible electroporation through a multi-electrode catheter may enable pulmonary vein isolation in as few as one shock per vein, which may produce much shorter procedure times compared to sequentially positioning a radiofrequency (RF) ablation tip around a vein.

It should be understood that while the energization strategies are described as involving DC pulses, embodiments may use variations and remain within the spirit and scope of the disclosure. For example, exponentially-decaying pulses, exponentially-increasing pulses, and combinations may be used.

Further, it should be understood that the mechanism of cell destruction in electroporation is not primarily due to heating effects, but rather to cell membrane disruption through application of a high-voltage electric field. Thus, electroporation may avoid some possible thermal effects that may occur when using radio frequency (RF) energy. This “cold therapy” thus has desirable characteristics.

With this background, and now referring again to FIG. 1, system 10 includes a catheter electrode assembly 12 including at least one catheter electrode. Electrode assembly 12 is incorporated as part of a medical device such as a catheter 14 for electroporation therapy of tissue 16 in a body 17 of a patient. In the illustrative embodiment, tissue 16 includes heart or cardiac tissue. It should be understood, however, that embodiments may be used to conduct electroporation therapy with respect to a variety of other body tissues.

FIG. 1 further shows a plurality of return electrodes designated 18, 20, and 21, which are diagrammatic of the body connections that may be used by the various sub-systems included in overall system 10, such as an electroporation generator 26, an electrophysiology (EP) monitor such as an ECG monitor 28, and a localization and navigation system 30 for visualization, mapping, and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode, and may include split patch electrodes (as described herein). In other embodiments, return electrodes 18, 20, and 21 may be any other type of electrode suitable for use as a return electrode including, for example, one or more catheter electrodes. Return electrodes that are catheter electrodes may be part of electrode assembly 12 or part of a separate catheter or device (not shown). System 10 may further include a main computer system 32 (including an electronic control unit 50 and data storage-memory 52), which may be integrated with localization and navigation system 30 in certain embodiments. System 32 may further include conventional interface components, such as various user input/output mechanisms 34A and a display 34B, among other components.

Electroporation generator 26 is configured to energize the electrode element(s) in accordance with an electroporation energization strategy, which may be predetermined or may be user-selectable. For electroporation-induced primary necrosis therapy, generator 26 may be configured to produce an electric current that is delivered via electrode assembly 12 as a pulsed electric field in the form of short-duration DC pulses (e.g., a nanoseconds to several milliseconds duration, or any duration suitable for electroporation) between closely spaced electrodes capable of delivering an electric field strength (i.e., at the tissue site) of about 0.1 to 1.0 kV/cm. The amplitude and pulse duration needed for irreversible electroporation are inversely related.

Electroporation generator 26, sometimes also referred to herein as a DC energy source, is a biphasic electroporation generator 26 configured to generate a series of DC energy pulses that all produce current in two directions. In other embodiments, electroporation generator is a monophasic or polyphasic electroporation generator. In some embodiments, electroporation generator 26 is configured to output energy in DC pulses at selectable energy levels, such as fifty joules, one hundred joules, two hundred joules, and the like. Other embodiments may have more or fewer energy settings and the values of the available setting may be the same or different. For successful electroporation, some embodiments utilize the two hundred joule output level. For example, electroporation generator 26 may output a DC pulse having a peak magnitude from about 300 Volts (V) to about 3,200 V at the two hundred joule output level. Other embodiments may output any other suitable positive or negative voltage.

In some embodiments, a variable impedance 27 allows the impedance of system 10 to be varied to limit arcing. Moreover, variable impedance 27 may be used to change one or more characteristics, such as amplitude, duration, pulse shape, and the like, of an output of electroporation generator 26. Although illustrated as a separate component, variable impedance 27 may be incorporated in catheter 14 or generator 26.

With continued reference to FIG. 1, as noted above, catheter 14 may include functionality for electroporation and in certain embodiments also additional ablation functions (e.g., RF ablation). It should be understood, however, that in those embodiments, variations are possible as to the type of ablation energy provided (e.g., cryoablation, ultrasound, etc.).

In the illustrative embodiment, catheter 14 includes a cable connector or interface 40, a handle 42, and a shaft 44 having a proximal end 46 and a distal 48 end. Catheter 14 may also include other conventional components not illustrated herein such as a temperature sensor, additional electrodes, and corresponding conductors or leads. Connector 40 provides mechanical and electrical connection(s) for cable 56 extending from generator 26. Connector 40 may include conventional components known in the art and as shown is disposed at the proximal end of catheter 14.

Handle 42 provides a location for the clinician to hold catheter 14 and may further provide means for steering or the guiding shaft 44 within body 17. For example, handle 42 may include means to change the length of a guidewire extending through catheter 14 to distal end 48 of shaft 44 or means to steer shaft 44. Moreover, in some embodiments, handle 42 may be configured to vary the shape, size, and/or orientation of a portion of the catheter, and it will be understood that the construction of handle 42 may vary. In an alternate embodiment, catheter 14 may be robotically driven or controlled. Accordingly, rather than a clinician manipulating a handle to advance/retract and/or steer or guide catheter 14 (and shaft 44 thereof in particular), a robot is used to manipulate catheter 14. Shaft 44 is an elongated, tubular, flexible member configured for movement within body 17. Shaft 44 is configured to support electrode assembly 12 as well as contain associated conductors, and possibly additional electronics used for signal processing or conditioning. Shaft 44 may also permit transport, delivery and/or removal of fluids (including irrigation fluids and bodily fluids), medicines, and/or surgical tools or instruments. Shaft 44 may be made from conventional materials such as polyurethane and defines one or more lumens configured to house and/or transport electrical conductors, fluids or surgical tools, as described herein. Shaft 44 may be introduced into a blood vessel or other structure within body 17 through a conventional introducer. Shaft 44 may then be advanced/retracted and/or steered or guided through body 17 to a desired location such as the site of tissue 16, including through the use of guidewires or other means known in the art.

In some embodiments, catheter 14 is a grid catheter having catheter electrodes (not shown in FIG. 1) distributed at the distal end of shaft 44. In some embodiments, catheter 14 has sixteen catheter electrodes. In other embodiments, catheter 14 includes ten catheter electrodes, twenty catheter electrodes, or any other suitable number of electrodes for performing electroporation. In some embodiments, the catheter electrodes are ring electrodes, such as platinum ring electrodes. Alternatively, the catheter electrodes may be any other suitable type of electrodes, such as partial ring electrodes or electrodes printed on a flex material. In various embodiments, the catheter electrodes have lengths of 1.0 mm, 2.0 mm, 2.5 mm, and/or any other suitable length for electroporation.

Localization and navigation system 30 may be provided for visualization, mapping and navigation of internal body structures. Localization and navigation system 30 may include conventional apparatus known generally in the art. For example, localization and navigation system 30 may be substantially similar to the EnSite Precision™ System, commercially available from Abbott Laboratories, and as generally shown in commonly assigned U.S. Pat. No. 7,263,397 titled “Method and Apparatus for Catheter Navigation and Location and Mapping in the Heart”, the entire disclosure of which is incorporated herein by reference. In another example, localization and navigation system 30 may be substantially similar to the EnSite X™ System, as generally shown in U.S. Pat. App. Pub. No. 2020/0138334 titled “Method for Medical Device Localization Based on Magnetic and Impedance Sensors”, the entire disclosure of which is incorporated herein by reference. It should be understood, however, that localization and navigation system 30 is an example only, and is not limiting in nature. Other technologies for locating/navigating a catheter in space (and for visualization) are known, including for example, the CARTO navigation and location system of Biosense Webster, Inc., the Rhythmia® system of Boston Scientific Scimed, Inc., the KODEX® system of Koninklijke Philips N.V., the AURORA® system of Northern Digital Inc., commonly available fluoroscopy systems, or a magnetic location system such as the gMPS system from Mediguide Ltd. In this regard, some of the localization, navigation and/or visualization system would involve a sensor be provided for producing signals indicative of catheter location information, and may include, for example one or more electrodes in the case of an impedance-based localization system, or alternatively, one or more coils (i.e., wire windings) configured to detect one or more characteristics of a magnetic field, for example in the case of a magnetic-field based localization system. As yet another example, system 10 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety.

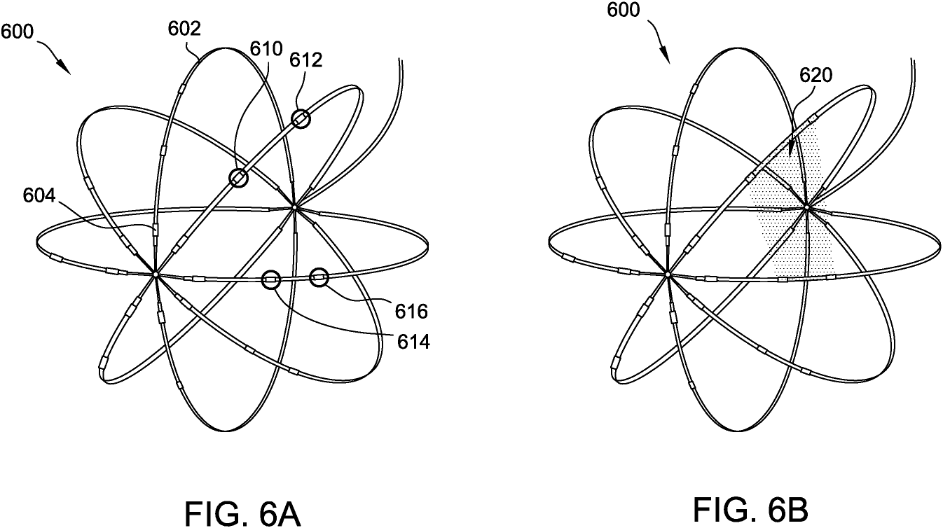

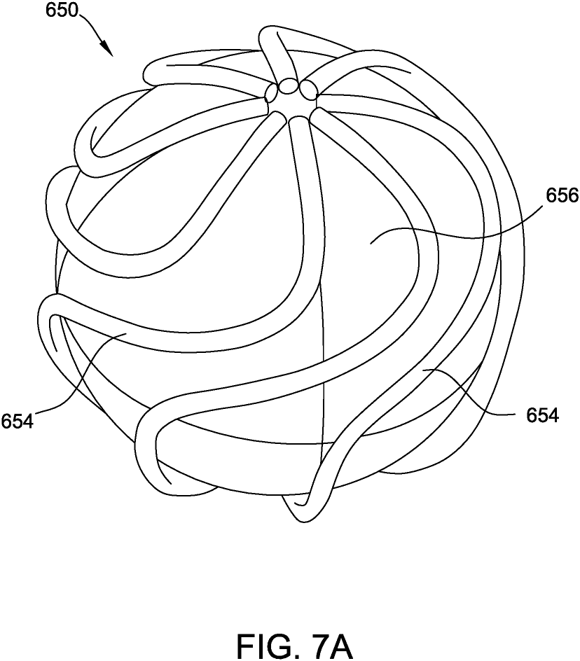

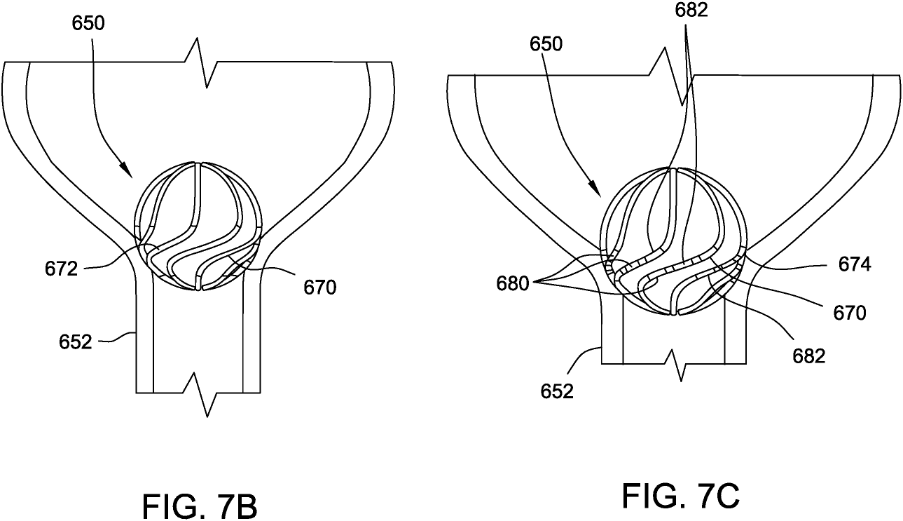

In at least some of the embodiments described herein, a catheter includes an array of electrodes that define one or more pixels. The array of electrodes may be arranged, for example, on a grid catheter (e.g., as shown in FIG. 2-5B) or on a basket catheter (e.g., as shown in FIGS. 6A-7C). Alternatively, the array of electrodes may be arranged on any suitable catheter assembly.

FIG. 2 is a side view of one embodiment of a grid assembly 200 that may be used with catheter 14 in system 10. Those of skill in the art will appreciate that, in other embodiments, any suitable catheter may be used. In addition, those of skill in the art will appreciate that, although the embodiments disclosed herein are discussed in the context of a grid catheter, the methods and systems described herein may be implemented using any suitable catheter (e.g., fixed loop catheters, basket catheters, linear catheters (for example, a catheter including a single spline), etc.). As shown in FIG. 2, grid assembly 200 is coupled to a distal section 202 of shaft 44.

Grid assembly 200 includes a plurality of splines 204 extending from a proximal end 206 to a distal end 208. Each spline 204 includes a plurality of electrodes 210. In the embodiment shown in FIG. 2, grid assembly 200 includes four splines 204, and each spline 204 includes four electrodes 210, such that electrodes 210 form a grid configuration. Accordingly, grid assembly 200 provides a four by four grid of electrodes 210. In one embodiment, the spacing between each pair of adjacent electrodes 210 is approximately 4 millimeters (mm) such that the dimensions of the grid of electrodes 210 are approximately 12 mm × 12 mm. Alternatively, grid assembly 200 may include any suitable number of splines 204, any suitable number of electrodes 210, and/or any suitable arrangement of electrodes 210. For example, in some embodiments, the spacing between each pair of adjacent electrodes is approximately 2 millimeters (mm). Further, in some embodiments, grid assembly 200 may include, for example, fifty-six electrodes arranged in a 7 × 8 grid.

Using grid assembly 200, lesions may be generated at individual electrodes 210 using a monopolar approach (e.g., by applying a voltage between individual electrodes 210 and a return patch), or generated between pairs of electrodes 210 using a bipolar approach. Lesions may be generating within an anatomy by selectively energizing electrodes in a particular configuration and/or pattern (e.g., including energizing individual electrodes 210 independent of one another, or energizing multiple electrodes 210 simultaneously).

FIG. 3 is an image 300 showing grid assembly 200 positioned within a left atrium 302 of a patient’s heart. As shown in FIG. 3, grid assembly 200 covers a relatively wide area of the heart. The width of this area is generally larger than that needed to perform pulmonary vein isolation (PVI). Accordingly, to perform a successful PVI ablation, it may be possible to only energize a portion of grid assembly 200.

Similarly, if a specific target (e.g., a 4 mm × 4 mm area, or any suitable size area) needs to be ablated, grid assembly 200 need only be placed somewhere over that spot, and electrodes 210 located proximate the specific target may be selectively activated. As a result, grid assembly 200 may be navigated with less precision (as compared to catheters with an electrode array having a smaller footprint.) For example, using grid assembly 200, a target may be ablated within an accuracy of 4 mm with only a placement accuracy of 12 mm.

To selectively energize electrodes 210 on grid assembly 200 that are proximate a target, a relatively high-precision mapping system is desirable (i.e., to accurately determine to position of grid assembly 200 relative to the tissue to be ablated). For example, the mapping system may be used to generate a visualization of the tissue, and a user can view the visualization to determine which electrodes 210 to selectively energize. Electrodes 210 may be selected by the user using a graphical user interface (GUI) (e.g., displayed on display 34B (shown in FIG. 1)).

In another embodiment, the user may draw or otherwise select one or more desired ablation or lesion locations on a surface of a displayed geometry (e.g., displayed on display 34B). The selected locations may be, for example, a line path representing a PVI or a point target representing a focal ablation. Once the user has selected one or more locations, grid assembly 200 may be navigated proximate the one or more locations. While grid assembly 200 is being navigated, system 10 may automatically detect (e.g., using mapping technology as described herein) when at least a portion of grid assembly 200 (e.g., one or more pixels, as described below) covers the one or more selected locations, and notify the user accordingly. At that point, system 10 may automatically determine which of electrodes 210 should be energized to achieve the desired ablation.

In addition, in some embodiments, a projected lesion pattern may be computed and displayed on the geometry (e.g., displayed on display 34B). This enables a user to visualize the lesion pattern, which may be relatively complex, depending on the energization scheme.

Further, in some embodiments, catheter 14 may be pulled relatively slowly (e.g., at a speed of approximately 1 to 10 millimeters per second) across a target ablation or lesion location. As catheter 14 is pulled, system 10 may automatically and continuously determine which electrodes 210 to energize. The combination of pulling catheter 14 and selectively activating electrodes 210 enables generating a continuous painted lesion. In some embodiments, system 10 may also track which targets have been ablated, and which targets still need to be ablated until all targets have been ablated. Accordingly, using grid assembly 200 in conjunction with a sophisticated mapping system enables a user to quickly and easily ablate one or more target locations.

Using bipolar delivery patterns, a plurality of different energization patterns are available using grid assembly 200. That is, in the systems and methods described herein, each electrode 210 may selectively function as a positive electrode, a negative electrode, or an inactive electrode.

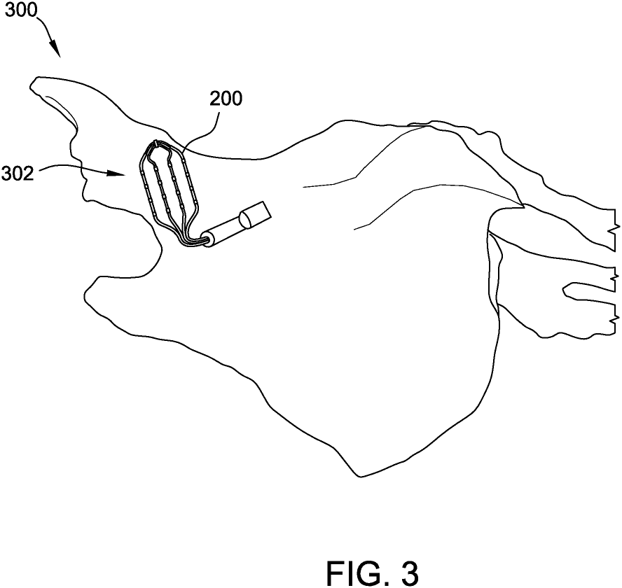

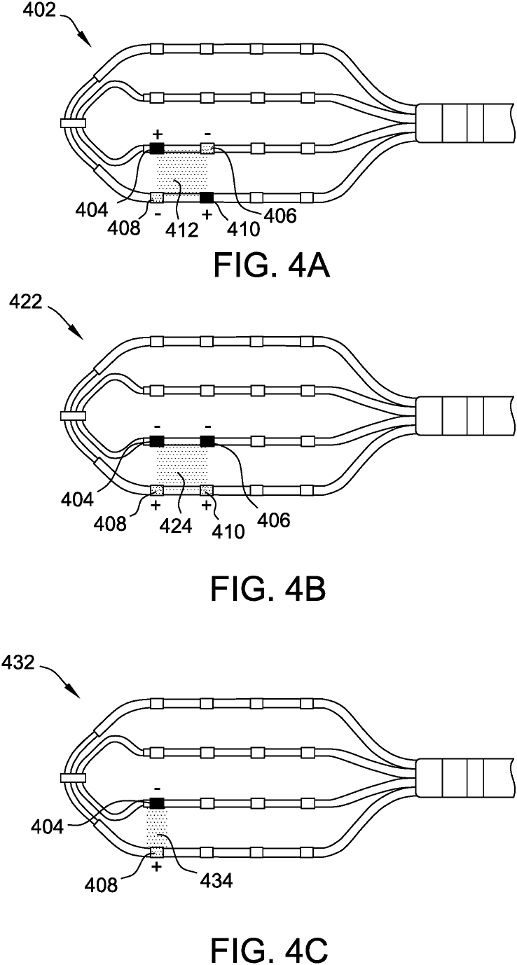

For example, FIGS. 4A-4C illustrate a plurality of example energization patterns using grid assembly 200. FIG. 4A illustrates a first energization pattern 402. In first energization pattern 402, a first electrode 404 functions as a negative electrode, a second electrode 406 functions as a positive electrode, a third electrode 408 functions as a positive electrode, a fourth electrode 410 functions as a negative electrode, and remaining electrodes 210 are inactive. With grid assembly 200 contacting tissue, first energization pattern 402 would ablate tissue proximate a first region 412.

FIG. 4B illustrates a second energization pattern 422. In second energization pattern 422, first electrode 404 functions as a negative electrode, second electrode 406 functions as a negative electrode, third electrode 408 functions as a positive electrode, fourth electrode 410 functions as a positive electrode, and remaining electrodes 210 are inactive. With grid assembly 200 contacting tissue, second energization pattern 422 would ablate tissue proximate a second region 424.

FIG. 4C illustrates a third energization pattern 432. In third energization pattern 432, first electrode 404 functions as a negative electrode, third electrode 408 functions as a positive electrode, and remaining electrodes 210 are inactive. With grid assembly 200 contacting tissue, third energization pattern 432 would ablate tissue proximate a third region 434.

For grid assembly 200, each 4 mm × 4 mm region defined by four electrodes 210 (e.g., first region 412 and second region 424) can be thought of as a pixel. Accordingly, grid assembly 200 includes a 3 × 3 grid of pixels. Each pixel can be selectively turned on or off (i.e., by energizing the four electrodes 210 corresponding to that pixel). Further, a region between two electrodes 210 (e.g., third region 434) can be thought of as a half-pixel. Third region 434 is a “vertical” half-pixel defined by two electrodes 210 that are on different splines 204. Those of skill in the art will appreciate that “horizontal” half-pixels may be defined by two adjacent electrodes 210 on the same spline 204, and “diagonal” half-pixels may be defined by two electrodes 210 on different splines 204 that are offset from one another (e.g., first electrode 404 and fourth electrode 410).

In the embodiments of FIGS. 4A-4C, a pixel is defined by four electrodes 410. Alternatively, a pixel may be defined by a different number of electrodes (e.g., three or five electrodes) in some embodiments.

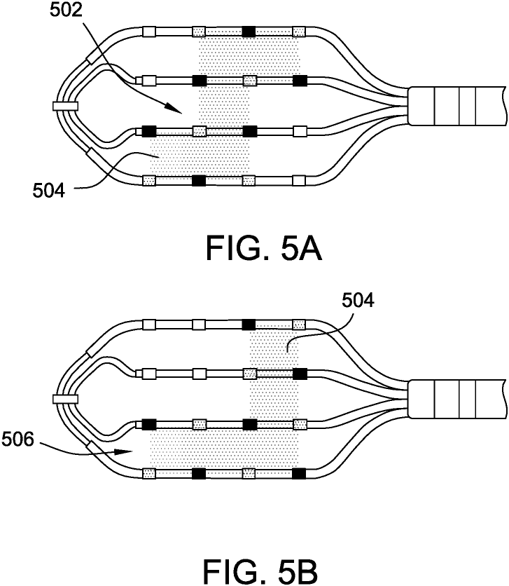

One or more pixels and/or half-pixels may be combined to form different energization patterns, as desired. FIGS. 5A and 5B illustrate two example energization patterns that may be implemented using grid assembly 200.

More specifically, FIG. 5A illustrates a first energization pattern 502. As shown in FIG. 5A, first energization pattern 502 is formed by five pixels 504 that combine to form an S-shaped pattern. FIG. 5B illustrates a second energization pattern 506. As shown in FIG. 5B, second energization pattern 506 is formed by five pixels 504 that combine to perform a L-shaped pattern. Those of skill in the art will appreciate that patterns 502 and 504 are merely examples, and that a wide variety of different patterns may be generated by combining one or more pixels and/or half pixels on grid assembly 200. Further, the pixels need not be contiguous with one another (i.e., at least some pixels may be separated by a gap).

Although the embodiments described herein are discussed in the context of IRE/PFA, those of skill in the art will appreciate that the methods and systems described herein may also be utilized for RF ablation applications.

Further, those of skill in the art will appreciate that the techniques described herein may be implemented with catheter configurations other than grid assembly 200. For example, FIGS. 6A and 6B are perspective views of one embodiment of a basket assembly 600 including a plurality of splines 602 that form a basket, each spline including a plurality of electrodes 604. Similar to grid assembly 200, pixels can be defined by sets of electrodes 604. For example, a first electrode 610, second electrode 612, third electrode 614, and fourth electrode 616 define a pixel 620 (shown in FIG. 6B). Other catheter configurations (e.g., spiral or linear catheters) may also utilize similar implementations. For a bipolar delivery scheme, at least two electrodes would be energized (at least one positive and at least one negative). For a monopolar delivery scheme, only one electrode need be energized (although for either polarity scheme, multiple electrodes may be energized).

FIGS. 7A-7C are views of another embodiment of a basket assembly 650 that may be used with the electrode energization techniques described herein. Specifically, FIG. 7A is a perspective view of basket assembly 650, and FIGS. 7B and 7C are side views of basket assembly 650 positioned within a pulmonary vein 652.

Basket assembly 650 includes a plurality of splines 654 that form a basket. In this embodiment, each spline 654 has a generally sigmoidal shape. The sigmoidal shape of splines 654 results in adjacent splines 654 maintaining roughly the same distance between one another along the length of splines 654, which may improve lesion quality. In this embodiment, basket assembly 650 includes eight splines 654. Alternatively, basket assembly 650 may include any suitable number of splines 654.

As shown in FIG. 7A, basket assembly 650 may include a selectively inflatable balloon 656 positioned in an interior of the basket. Balloon 656 may facilitate supporting splines 654 (e.g., when splines are pressed against tissue). In some embodiments, balloon 656 is omitted. Additional detail regarding basket assemblies with sigmoidal-shaped splines may be found in International Application No. PCT/US20/36410 entitled ELECTRODE BASKET HAVING HIGH-DENSITY CIRCUMFERENTIAL BAND OF ELECTRODES, filed on Jun. 5, 2020, and U.S. Provisional Patent Application No. 62/861,135, entitled ELECTRODE BASKET HAVING HIGH-DENSITY CIRCUMFERENTIAL BAND OF ELECTRODES, filed on Jun. 13, 2019, the disclosures of which are incorporated herein by reference in their entirety.

Each spline 654 include at least one electrode 670 that is selectively energizable using the systems and methods disclosed herein. For example, FIG. 7B shows one elongated electrode 672 on each spline 654, whereas FIG. 7C shows a plurality of individual electrodes 674 on each spline 654. Electrodes 670 are generally located on a distal portion of basket assembly 650, to facilitate contacting tissue of pulmonary vein 652. Alternatively, any suitable configuration of electrodes 670 may be used.

Similar to the embodiments described previously, sets of electrodes 670 on basket assembly 650 define pixels therebetween. Referring to FIG. 7B, in one electrode energization pattern, alternating splines 654 are assigned alternating polarities. That is, a spline 654 with a positive elongated electrode 672 is positioned between two splines 654 with negative elongated electrodes 672.

Referring to FIG. 7C, in another electrode energization pattern, individual electrodes 674 that are proximate one another on adjacent splines 654 are assigned the same polarity, but along each spline 654, individual electrodes 674 alternate polarity. For example, first individual electrodes 680 are positive, and second individual electrodes 682 are negative. Alternatively, any suitable electrode energization scheme may be used.

As noted above, a mapping system (e.g., localization and navigation system 30 (shown in FIG. 1)) may be used to detect a location of the catheter. For example, the mapping system may continuously check whether the catheter is in proximate to at least one location within a planned lesion set. The locations may be referred to as design points.

To determine whether one or more electrodes of the catheter are proximate a design point, the mapping system may continuously monitor distances between each electrode and the design point. When the mapping system determines a particular electrode is within a predetermined distance of the design point (e.g., an expected radius of a planned lesion), the mapping system detects that the electrode is proximate the design point. When the mapping system detects that an electrode is proximate the design point, the electrode and/or design point may be highlighted or otherwise emphasized on a display shown to a user (e.g., on display 34B).

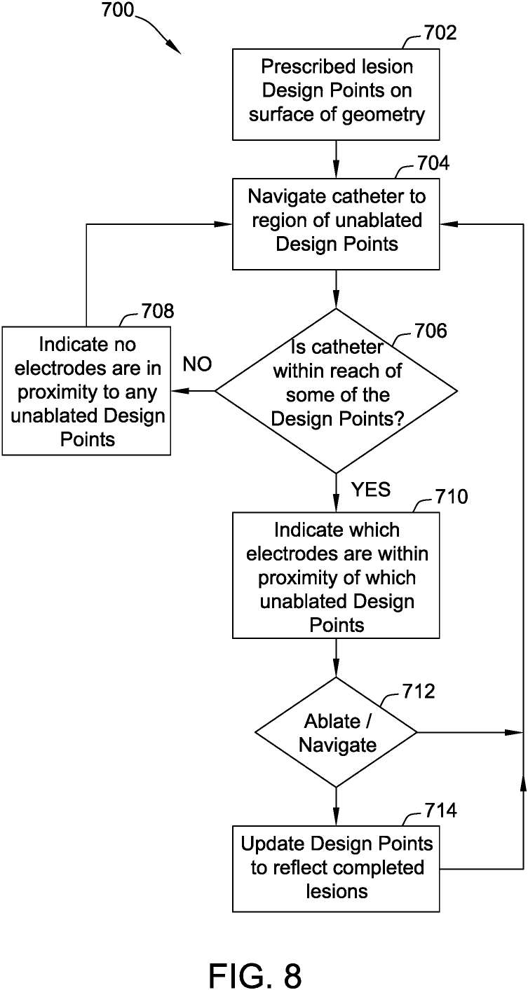

For example, FIG. 8 is a flowchart of an example method 700 for generating a lesion set using, for example, system 10 (shown in FIG. 1). Method 700 includes prescribing (i.e., identifying) one or more lesion design points on the surface of a geometry at block 702. The design points may be identified, for example, by a user operating a GUI.

Flow proceeds to block 704, at which the catheter is navigated to a region including one or more unablated design points (e.g., design points identified at block 702). Flow proceeds to block 706, and the system determines whether any electrodes are proximate any of the unablated design points. If not, flow proceeds to block 708, and the system indicates that no electrodes are proximate any of the unablated design points (e.g., by displaying a notification on the GUI), and flow returns to block 704. If, at block 706, at least one electrode is proximate at least one unablated design point, flow proceeds to block 710.

At block 710, the system indicates which electrodes are within proximity of which unablated design points (e.g., on GUI). Then, flow proceeds to block 712, and the user can choose to ablate the unablated design points (e.g., by selecting one or more electrodes to energize using the GUI) or continue to navigate the catheter. If the user decides to navigate the catheter, flow returns to block 704. If the user decides to perform ablation, flow proceeds to block 714, and the system updates the design points to reflect which design points have been ablated (e.g., by displaying that information on GUI). From block 714, flow returns to block 704.

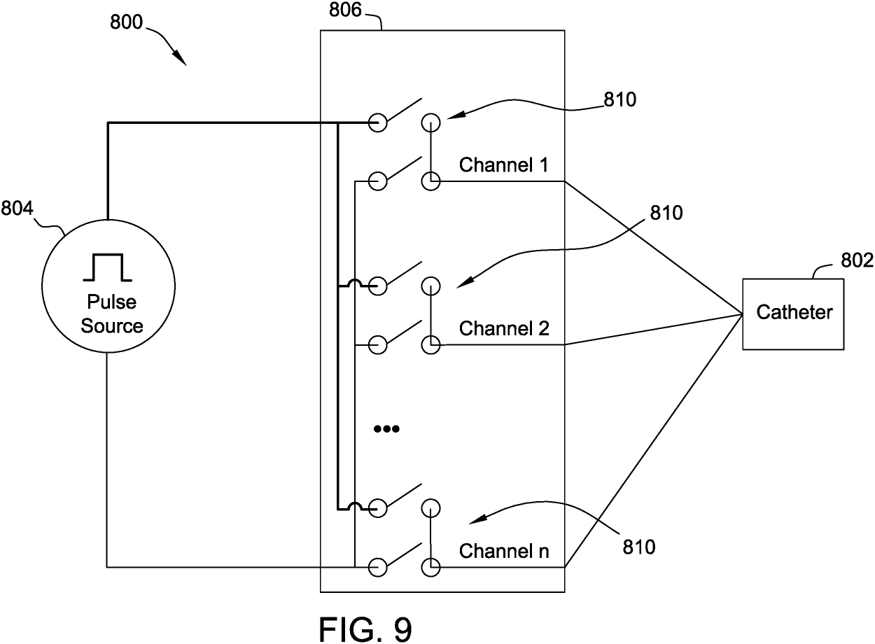

As described herein, electrodes on a catheter are selectively energized to generate different patterns. FIG. 9 is a schematic diagram of one embodiment of a switching architecture 800 that may be used to selectively energize electrodes on a catheter 802. Specifically, switching architecture includes a catheter 802, a pulse source 804, and a switching unit 806 coupled between catheter 802 and pulse source 804.

Pulse source 804 generates energy pulses to be applied by the electrodes (not shown) on catheter 802. Further, switching unit 806 includes a plurality of switching circuits 810 for selectively delivering energy pulses from pulse source 804 to the electrodes. In this embodiment, switching unit 806 includes a switching circuit 810 (and corresponding channel) for each electrode. Each switching circuit 810 receives an energy pulse from pulse source 804 and, depending on a configuration of switches within switching circuit 810, delivers a positive pulse, a negative pulse, or no pulse to the corresponding electrode. Accordingly, by controlling switching circuits 810, the electrodes on catheter 802 are selectively energizable.

In the embodiments described herein, the electrodes of the electroporation catheter may be energized to deliver therapeutic pulses (e.g., to generate lesions) and/or to deliver diagnostic pulses (e.g., to assess potential arrythmia sites). For example, prior to delivering therapeutic pulses, using the systems and methods described herein, energy can be applied for a relatively short duration to cause electrical flow interruption (which assists clinicians in identifying arrythmia sites). As the effects of the short duration diagnostic application subside, longer term therapeutic pulses can be subsequently applied.

The field strength for diagnostic pulses generally needs to be below a level at which cardiac cells are damaged (e.g., below 400 V/cm in the cardiac tissue). The field strength is not directly controllable, but depends on the applied voltage, tissue impedance, and catheter design (e.g., electrode size and spacing). In one example, diagnostic pulses are delivered with a field strength between 25 V/cm and 200 V/cm.

The embodiments described herein are directed to an apparatus for controlling an electroporation catheter is provided. The electroporation catheter includes a distal end, a proximal end, at least one spline extending from the distal end to the proximal end, and a plurality of electrodes arranged on the at least one spline. The apparatus includes a pulse generator coupled to the electroporation catheter, and a computing device coupled to the pulse generator, the computing device operable to control the pulse generator to selectively energize the plurality of electrodes on the electroporation catheter to form an energization pattern.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader’s understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. An apparatus for controlling an electroporation catheter, the electroporation catheter including a distal end, a proximal end, at least one spline extending from the distal end to the proximal end, and a plurality of electrodes arranged on the at least one spline, the apparatus comprising:
a pulse generator coupled to the electroporation catheter; and
a computing device coupled to the pulse generator, the computing device operable to control the pulse generator to selectively energize the plurality of electrodes on the electroporation catheter to form an energization pattern.

2. The apparatus in accordance with claim 1, wherein the energization pattern includes at least one pixel, the at least one pixel defined by three or four electrodes of the plurality of electrodes.

3. The apparatus in accordance with claim 1, wherein the energization pattern includes at least one half-pixel, the at least one half-pixel defined by two electrodes of the plurality of electrodes.

4. The apparatus in accordance with claim 1, wherein the electroporation catheter includes a grid assembly formed by a plurality of splines and the plurality of electrodes, the computing device operable to control the pulse generator to selectively energize the plurality of electrodes on the grid assembly.

5. The apparatus in accordance with claim 1, wherein the electroporation catheter includes a basket assembly formed by a plurality of splines and the plurality of electrodes, the computing device operable to control the pulse generator to selectively energize the plurality of electrodes on the basket assembly.

6. The apparatus in accordance with claim 1, wherein the computing device is operable to control the pulse generator to selectively energize the plurality of electrodes to deliver diagnostic pulses.

7. The apparatus in accordance with claim 1, wherein the computing device is operable to control the pulse generator to selectively energize the plurality of electrodes to deliver bipolar therapy.

8. The apparatus in accordance with claim 1, wherein the computing device is operable to:
display a geometry;
compute a projected lesion pattern based on the energization pattern; and
display the projected lesion pattern on the geometry.

9. A method for controlling an ablation system including an ablation catheter, a pulse generator coupled to the ablation catheter, and a computing device coupled to the pulse generator, the method comprising:
identifying, using the computing device, at least one target ablation location on an anatomy of a patient;
tracking, using the computing device, movement of the ablation catheter through the patient, the ablation catheter including a plurality of electrodes;
determining, based on the tracking, using the computing device, that at least one electrode of the plurality of electrodes is proximate the at least one target ablation location; and
selectively energizing, using the pulse generator, the at least one electrode to ablate the at least one target ablation location.

10. The method in accordance with claim 9, wherein identifying at least one target ablation location comprises identifying the at least one target ablation location based on user input received at the computing device.

11. The method in accordance with claim 9, further comprising:
generating, using the computing device, a user notification that indicates the at least one electrode is proximate the at least one target ablation location; and
receiving, at the computing device, user input indicating that the at least one electrode should be selectively energized, wherein selectively energizing the at least one electrode comprises selectively energizing the at least one electrode in response to the user input.

12. The method in accordance with claim 9, wherein selectively energizing the at least one electrode comprises automatically energizing the at least one electrode in response to detecting that the at least one electrode is proximate the at least one target ablation location.

13. The method in accordance with claim 9, further comprising updating a graphical user interface to indicate that the at least one target ablation location has been ablated.

14. The method in accordance with claim 9, wherein the ablation catheter includes a plurality of splines and a plurality of electrodes arranged on the plurality of splines.

15. The method in accordance with claim 14, wherein the plurality of splines and the plurality of electrodes form a grid assembly.

16. The method in accordance with claim 14, wherein the plurality of splines and the plurality of electrodes form a basket assembly.

17. An apparatus for controlling an electroporation catheter, the electroporation catheter including a distal end, a proximal end, a plurality of splines extending from the distal end to the proximal end, and a plurality of electrodes arranged on the plurality of splines, the plurality of splines and the plurality of electrodes forming a grid assembly, the apparatus comprising:
a pulse generator coupled to the electroporation catheter; and
a computing device coupled to the pulse generator, the computing device operable to control the pulse generator to selectively energize the plurality of electrodes on the grid assembly to form an energization pattern.

18. The apparatus in accordance with claim 17, wherein the energization pattern includes at least one pixel, the at least one pixel defined by three or four electrodes of the plurality of electrodes on the grid assembly.

19. The apparatus in accordance with claim 17, wherein the energization pattern includes at least one half-pixel, the at least one half-pixel defined by two electrodes of the plurality of electrodes on the grid assembly.

20. The apparatus in accordance with claim 17, wherein the computing device is operable to control the pulse generator to selectively energize the plurality of electrodes on the grid assembly to deliver bipolar therapy.
